(19) United States
(12) Patent Application Publication
(10) Pub. No.: US 2021/0170342 A1
(43) Pub. Date: Jun. 10, 2021

(54) FUNCTIONALIZED POLYMERIC MEMBRANES FOR THE SEPARATION, RECOVERY, AND/OR PURIFICATION OF ANTIBODIES

(71) Applicant: King Abdullah University of Science and Technology, Thuwal (SA)

(72) Inventors: Giada SOLDAN, Thuwal (SA); Suzana NUNES, Thuwal (SA)

(21) Appl. No.: 16/770,899

(22) PCT Filed: Dec. 13, 2018
(86) PCT No.: PCT/IB2018/060039

§ 371 Date: Jun. 8, 2020

Related U.S. Application Data
(60) Provisional application No. 62/598,013, filed on Dec. 13, 2017.
(60) Provisional application No. 62/760,501, filed on Nov. 13, 2018.

(51) Int. Cl. B01D 69/02; B01D 71/80; B01D 71/02; B01D 71/28; B01D 15/38; B01D 15/34; B01D 15/26; C07K 1/16

(52) U.S. Cl. CPC B01D 69/02; B01D 71/80; B01D 71/022; B01D 71/28; B01D 2325/12; B01D 15/34; B01D 15/265; C07K 1/16; B01D 15/3804

(57) ABSTRACT

Embodiments of the present disclosure describe functionalized polymeric membranes including one or more dithiol compounds that extend from a nanoparticle provided on or near a surface and/or pores of a polymer material, wherein at least one thiol of the dithiol compound binds to the nanoparticle and at least one thiol of the dithiol compound is a free thiol. Embodiments of the present disclosure further describe methods of separating and/or recovering a purified antibody comprising contacting a feed stream containing an antibody and other biomolecules with a functionalized polymeric membrane to separate the antibody from the feed stream; and applying a reducing agent to release the antibody from the membrane and recover a purified antibody; wherein the functionalized polymeric membrane includes a plurality of free thiols selective to binding the antibody.

### BACKGROUND

Antibodies have great utility in a variety of industries. Antibodies may be used in the development of pharmaceuticals, therapeutic agents for inflammatory, cardiovascular, and infectious diseases, and oncological treatments for advanced diagnosis. The primary drawback of the availability of antibodies is the need to achieve high purity levels to meet high efficiency and safety standards.

Antibodies are typically produced together with a mixture of other proteins, enzymes, or DNA. Current purification technologies rely on a series of steps to separate the various species and isolate the desired product. Many separation techniques are applied in sequence, moving from centrifugation to ultrafiltration, microfiltration and chromatography. These long multi-step processes for the separation and purification of antibodies account for up to 90% of production costs in the downstream processing industry.

In addition, antibodies are typically characterized as large, Y-shaped proteins. Each tip of the Y-shaped antibodies generally includes two active sites, each of which include a paratope that is specific to binding with a particular epitope on an antigen. The paratopes contain heavy chains and light chains including variable regions and constant regions. The variable regions generally contribute to or are responsible for binding to antigens. Such active sites of the antibody, however, cannot get in contact with any surface otherwise the overall activity of the antibody may be compromised.

Accordingly, it would be desirable to reduce the cost of separating and purifying antibodies, without compromising the overall quality of the purified product.

### SUMMARY

In general, embodiments of the present disclosure describe functionalized polymeric membranes, and methods of separating, recovering, and/or purifying antibodies using the functionalized polymeric membranes.

Accordingly, embodiments of the present disclosure describe a functionalized polymeric membrane including one or more dithiol compounds that extend from a nanoparticle provided on or near a surface and/or pores of a polymer material, wherein at least one thiol of the dithiol compound binds to the nanoparticle and at least one thiol of the dithiol compound is a free thiol.

Embodiments of the present disclosure further describe a method of separating and/or recovering a purified antibody comprising contacting a feed stream containing an antibody and other biomolecules with a functionalized polymeric membrane to separate the antibody from the feed stream; and applying a reducing agent to release the antibody from the membrane and recover a purified antibody; wherein the functionalized polymeric membrane includes a plurality of free thiols selective to binding the antibody.

The details of one or more examples are set forth in the description below. Other features, objects, and advantages will be apparent from the description and from the claims.

### BRIEF DESCRIPTION OF DRAWINGS

This written disclosure describes illustrative embodiments that are non-limiting and non-exhaustive. In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

Reference is made to illustrative embodiments that are depicted in the figures, in which:

FIG. 1 is a flowchart of a method of separating and/or recovering a purified antibody, according to one or more embodiments of the present disclosure.

FIG. 2 is a process schematic diagram showing the separation of antibodies using a functionalized polymeric membrane, according to one or more embodiments of the present disclosure. As shown in FIG. 2, a feed stream including a mixture of proteins (e.g., BSA) and antibodies (IgG) were injected. Due to differences in sizes, the BSA almost completely permeated through the membrane, while IgG was retained (e.g., via a separation step). The functionalization provided on the surface of the membrane allowed for the formation of a disulfide bond between the IgG and the dithiol compound, which temporarily retained the antibodies. In this way, the product can be and was later recovered in a purified state.

FIGS. 3A-3B are schematic diagrams showing (A) the two sites for reaction, which are represented as including a thiolate of the IgG and a thiolate of a dithiol ligand surrounding the gold nanoparticles and (B) an S-thiolation process occurring between the two species, bringing to the oxidation of the two sites, forming a disulfide bond, according to one or more embodiments of the present disclosure.

FIG. 4 shows an image of the polymeric membrane, according to one or more embodiments of the present disclosure.

FIG. 5 shows an image of the functionalized polymeric membrane, according to one or more embodiments of the present disclosure.

FIG. 6 is a table summarizing the adsorption of IgG (e.g., the mass adsorbed, % mass adsorbed, and adsorption capacity of the membranes), according to one or more embodiments of the present disclosure.

FIG. 7 is a schematic diagram illustrating the desorption process using DTT, according to one or more embodiments of the present disclosure.

### DETAILED DESCRIPTION

The invention of the present disclosure relates to functionalized polymeric membranes for one or more of the separation, recovery, and purification of antibodies. In particular, the invention of the present disclosure relates to polymeric membranes functionalized with nanoparticles and polythiol compounds, such as dithiol compounds. For example, the functionalized polymeric membranes described herein may include one or more dithiol compounds that extend from a nanoparticle provided on or near a surface and/or pores of a polymer material. The functionalized polymeric membrane may include a plurality of such nanoparticles from which the one or more dithiol compounds may extend. The dithiol compounds may extend from a nanoparticle through a thiol such that the other thiol is a free thiol. For example, at least one thiol of the one or more dithiol compounds may bind to the nanoparticle and at least one thiol of the one or more dithiol compounds may be a free thiol. In this way, the functionalized polymeric membranes provide a free thiol that is exposed or otherwise available to interact and/or react with, for example, antibodies.

The functionalized polymeric membranes described herein may be used for the separation of antibodies from a feed stream including one or more other chemical species, such as biomolecules, among others. In many embodiments, the antibodies may be selectively separated from the other chemical species due to differences in size. For example, a feed stream may be contacted with the membrane such that the functionalized polymeric membrane retains the antibodies, while the other chemical species completely or nearly completely permeate through the membrane. In addition or in the alternative, the functionalized polymeric membrane may adsorb or bind to a specific site of the antibody, resulting in an unprecedented specificity of binding. For example, the dithiol compounds of the membrane may bind to heavy chains of the antibodies through an exposed or free thiol. The adsorption is selective and specific such that antibodies are adsorbed to the exclusion or substantial exclusion of the other chemical species, without compromising the overall activity of the antibody. The antibodies once separated may then be desorbed or released using a reducing agent to recover purified antibodies.

### Definitions

The terms recited below have been defined as described below. All other terms and phrases in this disclosure shall be construed according to their ordinary meaning as understood by one of skill in the art.

As used herein, “antibody” is used in a broad sense and may refer to immunoglobulins, monoclonal antibodies (including full-length monoclonal antibodies), polyclonal antibodies, multi-specific antibodies, and antibody fragments. The “antibody” may refer to a class or subclass or fragment. For example, the major classes of antibodies may include one or more of IgA, IgD, IgE, IgG, and IgM, with several of these classes divided into subclasses, such as one or more of IgG1, IgG2, IgG3, IgG4, IgA1, and IgA2.

The “antibody” may refer to a single antibody, one or more antibodies, and/or a plurality of antibodies. For example, the term “antibody” may refer to one or more of a single antibody in a single class and/or single subclass, one or more antibodies in a single class and/or single subclass, one or more antibodies in multiple classes or multiple subclasses, a plurality of antibodies in a single class and/or single subclass, and a plurality antibodies in multiple classes and/or multiple subclasses.

As used herein, “monoclonal antibody” refers to an antibody obtained from a population of substantially homogeneous antibodies. For example, the individual antibodies of the population may be identical except for possible natural or unnatural mutations, which may be present in low or minor amounts. The term “monoclonal” shall not be construed as requiring production of the antibody by any particular method.

As used herein, “Fc region” refers to the fragment, crystallizable region of an antibody. The Fc region generally refers to the base of Y-shaped antibodies, which is generally located at or near a bottom portion of the antibody, and includes two heavy chains, which contribute constant domains.

As used herein, “Fab region” refers to the fragment, antigen-binding region of an antibody. The Fab region generally refers to the arms of Y-shaped antibodies, which are generally located at or near a top of the antibody, and includes a heavy chain and a light chain in each of the arms of Y-shaped antibodies. The Fab region generally includes a variable domain and a constant domain from each heavy and light chain of the antibody. Paratopes are located in the Fab region of antibodies.

As used herein, “heavy chain of an antibody” and similar or related terms generally refers to the Fc region of antibodies, such as the region provided at or near a base of Y-shaped antibodies. In many embodiments, for example, the functionalized polymeric membrane binds to a heavy chain in the Fc region of an antibody. In this way, antibodies may be separated, recovered, and/or purified from other chemical species without affecting, degrading, or otherwise compromising any activity of the antibody.

As used herein, “specificity” and/or “specific,” where used in a context of binding to or adsorbing an antibody for purposes of achieving a separation, generally refers to binding to a specific site of the antibody, such as a heavy chain in the Fc region of the antibody. For example, “specificity of binding,” “binding specificity,” “specific binding,” “binding to a specific site,” “specific adsorption,” “specifically adsorbing,” “adsorbing at a specific site,” and similar or related terms generally refers to binding to a heavy chain in the Fc region of the antibody.

As used herein, “dithiol compound” refers to any molecule, compound, and/or material including at least two thiols or thiol groups (R—SH).

As used herein, “thiolate” may refer to a deprotonated thiol or thiol group, such as RS⁻.

As used herein, “thiol” and/or “thiol group” may refer to an organosulfur compound with a carbon-bonded sulfhydryl group, such as R—SH, where R is any substituent including a carbon atom.

As used herein, “biomolecule” refers to any molecule of biological origin. For example, “biomolecule” may include organic biomolecules, such as one or more of steroids, amino acids, nucleotides, sugars, proteins, peptides, polypeptides, polynucleotides, complex carbohydrates, lipids, and other chemical compounds.

As used herein, “reducing agent” refers to any element, molecule, compound, and/or material capable of cleaving or breaking a bond, such as a disulfide bond.

As used herein, “applying” refers to contacting, such as bringing two or more components into physical contact, or immediate or close proximity.

As used herein, “contacting” refers to the act of touching, making contact, or of bringing to close or immediate proximity, including at the cellular or molecular level, for example, to bring about a physiological reaction, a chemical reaction, or a physical change (e.g., in solution, in a reaction mixture, in vitro, or in vivo). Contacting may refer to bringing two or more components in proximity, such as physically, chemically, electrically, or some combination thereof.

Embodiments of the present disclosure describe functionalized polymeric membranes for the separation, recovery, and/or purification of antibodies. In general, the functionalized polymeric membranes may comprise a polymer material, one or more nanoparticles, and one or more polythiol compounds. In many embodiments, the functionalized polymeric membranes may comprise one or more dithiol compounds that extend from a nanoparticle provided on or near a surface and/or pores of a polymer material. In an embodiment, at least one thiol of the dithiol compound may bind to the nanoparticle and at least one thiol of the dithiol compound may be a free thiol. In an embodiment, the functionalized polymeric membrane may include a plurality of such nanoparticles from which one or more dithiol compounds may extend.

The functionalized polymeric membrane may include a polymer material. The polymer material may be a polymeric membrane. The polymer material may include a porous structure, such as an asymmetric porous structure. The porous structure may include one or more of a porous support layer (e.g., a porous substructure) and a thin porous layer (e.g., a thin film and/or block copolymer film). For example, the porous structure may include a porous support layer covered by, or interconnected with, a thin porous layer. The thin porous layer may be an isoporous layer, wherein the diameters of the pores may be the same or substantially the same. The thin porous layer may include a uniform pore morphology with a highly ordered dense array of channels and/or pores aligned vertically or substantially vertically, or perpendicular or substantially perpendicular, to a surface of the thin porous layer. For example, the thin layer may include highly ordered pores containing cylinders or aligned interconnected spheres oriented perpendicular or substantially perpendicular to a surface of the thin layer. The pores of the porous structure may increase in size from the pores of the thin layer to the pores of the porous support layer.

The polymer material may include one or more block copolymers. The block copolymers may include one or more polymer blocks, such as one or more of a hydrophobic block and a hydrophilic block. In many embodiments, the polymer material may include one or more of polystyrene, poly-4-vinylpyridine, poly-2-vinylpyridine, polybutadiene, polyisoprene, poly(ethylene-stat-butylene), poly(ethylene-alt-propylene), polysiloxane, polyalkyleneoxide, poly-ε-caprolactone, polylactide, polyalkylmethacrylate, polymethacrylic acid, polyalkylacrylate, polyacrylic acid, polyhydroxyethylmethacrylate, polyacrylamide, poly-N-alkylacrylamide, polysulfone, polyaniline, polypyrrole, polytriazole, polyvinylimidazole, polytetrazole, polyethylenediamine, polyvinylalcohol, polyvinylpyrrolidone, polyoxadiazole, polyvinylsulfonic acid, polyvinylphosphonic acid, and polymers with quarternary ammonium groups. In a preferred embodiment, the polymer material includes a block copolymer, such as PS-b-P4VP.

The polymeric membrane may be functionalized with a nanoparticle, one or more nanoparticles, and/or a plurality of nanoparticles provided on or near a surface and/or pores of the polymer material. The one or more nanoparticles may be about uniformly and/or about randomly distributed on or near the surface and/or pores of the polymer material. A diameter or size of the one or more nanoparticles may be smaller than a pore size or pore diameter of the polymer material, such that the nanoparticles do not block or obstruct pores. The one or more nanoparticles may bind or attach to the surface and/or pores of the polymer material through one or more of covalent bonds, ionic bonds, dipole-dipole interactions, and hydrogen bonds, among others. In many embodiments, the one or more nanoparticles may bind or attach to the polymer material through covalent bonds.

The one or more nanoparticles may generally include any element, compound, or material capable of binding to the polymer material and/or the dithiol compound. The nanoparticles may be inorganic, organic, or a combination thereof. In many embodiments, the nanoparticles may include a metal capable of forming a metal-sulfur bond with a dithiol compound. For example, the nanoparticles may include one or more of gold, silver, and copper nanoparticles. In a preferred embodiment, the nanoparticles may include one or more gold nanoparticles. The gold nanoparticles may provide affinity, such as chemical and/or physical affinity, for increasing adsorption of antibodies, among other things. The gold nanoparticles may be biocompatible and may exhibit reversible binding behavior, as well as a high surface area-to-volume ratio, providing a large number of binding sites for the dithiol compounds. In addition, the gold nanoparticles may bind to thiol groups due to a strong interaction between gold and sulfur.

The polymeric membrane may be functionalized with one or more dithiol compounds. The dithiol compounds may be included to provide selectivity for antibodies and/or a specific binding or specific adsorption to a thiol or thiolate of a heavy chain of an antibody. The functionalized polymeric membrane may include one or more dithiol compounds that extend from a nanoparticle. Each of the one or more dithiol compounds may bind to a nanoparticle through a thiol or thiolate of the dithiol compound. For example, a dithiol compound may include at least one thiol that binds to a nanoparticle and at least one thiol that is a free thiol. A plurality of nanoparticles provided on or near a surface or pores of the polymer material may bind with one or more dithiol compounds (e.g., a plurality of dithiol compounds). In some embodiments, the functionalized polymeric membrane may include one or more nanoparticles that are not attached or bound to a dithiol compound. In general, the membrane includes at least one dithiol attached to at least one nanoparticle.

The free thiol may extend from the nanoparticle such that it is exposed or otherwise available to interact (e.g., react or bind) with a thiol or thiolate of a heavy chain of an antibody. In an embodiment, the free thiol of the dithiol compound selectively binds to an antibody. For example, the free thiol of the dithiol compound may bind to an antibody, without promoting any interaction or any substantial interaction with other biomolecules (e.g., a counter-protein, such as BSA). In an embodiment, the free thiol of the dithiol compound may specifically bind to a thiol of a heavy chain of the antibody. In this way, the functionalized polymeric membranes may adsorb or bind to an antibody through its heavy chains, without affecting or substantially affecting the activity of the antibody.

The dithiol compound may include at least two thiols, such as R—SH, or thiolates, such as RS⁻, where R is any substituent (e.g., organic substituent). The dithiol compound may include one or more of an aliphatic dithiol compound, aromatic dithiol compound, and oligomeric dithiol compound. In many embodiments, the dithiol compound may include one or more of 1,2-ethanedithiol, butanedithiol, 1,3-propanedithiol, 1,5-pentanedithiol, 2,3-dimercapto-1-propanol, dithioerythritol, 3,6-dioxa-1,8-octanedithiol, 1,8-octanedithiol hexanedithiol, dithiodiglycol, pentanedithiol, decanedithiol, 2-methyl 1,4 butanedithiol, bis-mercaptoethylphenyl methane, 1,9-nonanedithiol(1,9-dimercaptononane), glycol dimercaptoacetate, 1,2-benzenedithiol, 1,3-benzenedithiol, 1,4-benzenedithiol, 2,4,6-trimethyl-1,3-benzenedimethanethiol, durene-α1, α2-dithiol, 3,4-dimercaptotoluene, 4-methyl-1,2-benzenedithiol, 2,5-dimercapto-1,3,4-thiadiazole, 4,4′-thiobisbezenedithiol, and bis(4-mercaptophenyl)-2,2′-propane(bisphenol dithiol). In a preferred embodiment, the dithiol compound may include an aliphatic dithiol, such as 1,3-propanedithiol.

In an embodiment, the functionalized polymeric membrane may comprise one or more dithiol compounds that extend from a gold nanoparticle provided on or near a surface and/or pores of a block copolymer membrane, wherein at least one thiol of the dithiol compound binds to the nanoparticle and at least one thiol of the dithiol compound is a free thiol.

The functionalized polymeric membranes described herein may be used for one or more of the separation, recovery, and purification of an antibody or antibodies. Accordingly, embodiments of the present disclosure describe methods of separating an antibody, methods of recovering an antibody, and methods of purifying an antibody.

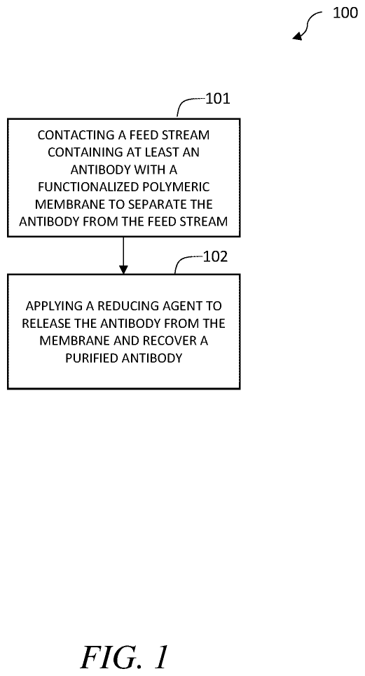

FIG. 1 is a flowchart of a method of separating and/or recovering a purified antibody. As shown in FIG. 1, the method 100 may comprise contacting 101 a feed stream containing at least an antibody and one or more other chemical species with a functionalized polymeric membrane to separate the antibody from the feed stream and applying 102 a reducing agent to release the antibody from the membrane and recover a purified antibody. The functionalized polymeric membrane may include any of the membranes described herein. In many embodiments, the functionalized polymeric membrane includes a plurality of free thiols selective for and/or specific to binding the antibody. In an embodiment, the one or more other chemical species includes one or more other biomolecules.

The step 101 includes contacting a feed stream containing at least an antibody and other biomolecules with a functionalized polymeric membrane to separate the antibody from the feed stream. In this step, the feed stream may be brought into physical contact, or immediate or close proximity to, the functionalized membrane. The contacting may proceed at any pH (e.g., a pH ranging from about 0 to about 14 and/or one or more of acidic, neutral, and basic). In many embodiments, the pH is selected and/or adjusted to facilitate interactions between one or more of the functionalized polymeric membrane, antibody, and other chemical species present in the feed stream. For example, the contacting may proceed at a pH such that the functionalized polymeric membrane and antibody have opposite charges, favoring attraction. The contacting may proceed at a pH such that the functionalized polymeric membrane and the other species present in the feed stream have the same charge, favoring repulsion. In many embodiments, the pH is less than about 8. In a preferred embodiment, the pH is about 7, about 6.5, and/or about 6.

The feed stream may further contain at least an antibody and one or more other chemical species. The antibody may be capable of forming a disulfide bond through a heavy chain of the antibody. In many embodiments, the feed stream may contain an antibody and one or more other chemical species. The one or more other chemical species may include one or more of biomolecules, biochemicals, and other chemical species (e.g., elements, molecules, compounds, materials, etc.) typically present or associated with an antibody to be purified. For example, the feed stream may include an antibody source, such as bulk unprocessed serum or blood, laboratory produced monoclonal antibodies, or other sources known in the art, among others. In many embodiments, the feed stream may include one or more of proteins, peptides, cells, cell debris, nucleic acids, endotoxins, and viruses, among other things. In an embodiment, the feed stream may include a monoclonal antibody, such as γ-immunoglobulin (IgG), and another protein or counter-protein, such as bovine serum albumin (BSA).

The functionalized polymeric membrane may exhibit an unprecedented selectivity for and/or specificity of binding to the antibody. The selectivity may be based on one or more of separation and adsorption. In particular, the functionalized polymeric membrane may selectively separate the antibody from the feed stream based on differences in size between the antibody and the other chemical species. For example, the other chemical species may permeate through the membrane sufficient to yield a retentate comprising or consisting essentially of the antibody and/or a permeate comprising or consisting essentially of the other chemical species. In addition or in the alternative, the functionalized polymeric membrane may selectively adsorb or bind to the antibody, to the exclusion or substantial exclusion of the other chemical species, with a high specificity of binding. For example, the antibody may be adsorbed through its heavy chains—not through its active sites (e.g., the active sites located at each tip near the top of Y-shaped antibodies)—without compromising the overall activity of the antibody.

While not wishing to be bound to a theory, it is suggested here that the selective adsorption and/or high specificity of binding of the antibody may be related to the formation of a disulfide bond between the antibody and the membrane in a process or reaction referred to as S-thiolation. In particular, the membranes may incorporate dithiol compounds to provide a functionalized polymeric membrane including at least a free thiol. The free thiol of the membrane may react with an exposed thiol of a heavy chain of the antibody to form the disulfide bond. In some embodiments, the reaction conditions (e.g., pH, etc.) may be selected or adjusted to provide reduced or partially reduced thiolates of the heavy chain of the antibody. For example, in an embodiment, the contacting proceeds at about a neutral pH to reduce or partially reduce thiolates of the heavy chain of the antibodies and expose —SH groups that react with the free thiols of the membrane to form the disulfide bond and adsorb the antibody.

The step 102 includes applying a reducing agent to release the antibody from the membrane and recover a purified antibody. In this step, the reducing agent may be applied sufficient to be brought into contact with, or immediate or close proximity to, the adsorbed antibodies and/or the functionalized polymeric membrane. The application of the reducing agent may be used to cleave or break disulfide bonds, thereby desorbing the antibody from the membrane. In many embodiments, desorption of the antibody may be achieved by reducing the most exposed disulfide bond, i.e., the disulfide bond connecting the antibody to the membrane through dithiol. Upon being desorbed or otherwise released, a purified antibody may be recovered.

The applying may proceed at a pH sufficient to form a reactive thiolate. For example, in some embodiments, the reducing agent may include, but is not limited to, dithiothreitol (DTT). The DTT exhibits a selectivity for “attacking” the disulfide bond and is biocompatible. In embodiments in which DTT is used, DTT may include thiols in a reduced state (—SH). In order to cleave or break disulfide bonds, DTT may need to be oxidized or deprotonated since generally only a negatively charged thiolate with the formula —S⁻ is reactive. Accordingly, in some embodiments, the applying may proceed at a pH of about 7 in order to oxidize DTT and form the desired reactive species. In other embodiments, the reducing agent may include any element, compound, or material that is capable of cleaving or breaking disulfide bonds (e.g., selective) and biocompatible.

The following Examples are intended to illustrate the above invention and should not be construed as to narrow its scope. One skilled in the art will readily recognize that the Examiners suggest many other ways in which the invention could be practiced. It should be understand that numerous variations and modifications may be made while remaining within the scope of the invention.

### Example 1

### Functionalized Polymeric Membrane for Separation and Recovery of Antibodies

Antibodies are usually produced together with a mixture of other proteins, enzymes or DNA, therefore current purification technologies deal with a series of different steps in order to separate the different species and isolate the desired product. Hence, many separation techniques are applied in sequence, moving from centrifugation to ultrafiltration, microfiltration and chromatography. The long process is responsible for the high costs generally associated with the downstream industry, therefore the current proposed technology aims at shortening that process and making the distribution of purified antibodies more available in the market.

The Example describes functionalized polymeric membranes that aim to reduce the long steps and high costs of downstream processing in the biotech and pharmaceutical industries. Specifically, a functionalized polymeric membrane was designed to purify monoclonal antibodies, where the targeted class of antibody was γ-ImmunoGlobulins, or IgG.

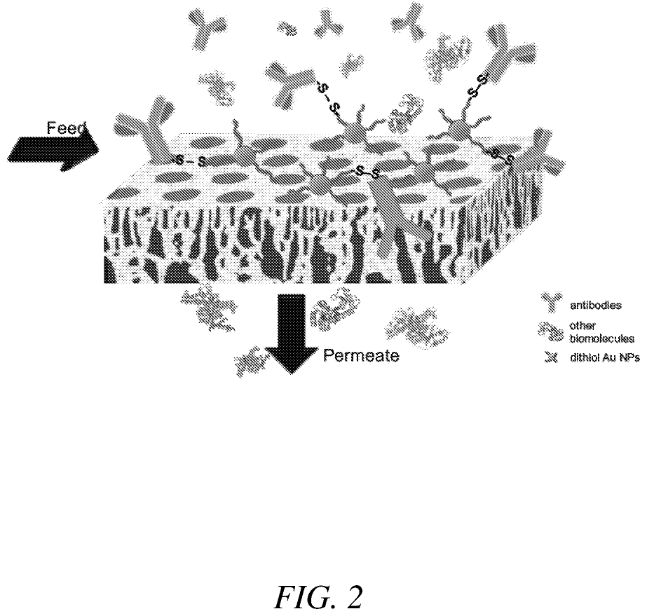

In particular, the Example describes a polymeric membrane, functionalized with gold nanoparticles and dithiol molecules, that selectively separated bovine serum albumin (BSA) from IgG, while allowing a specific binding of the antibody to the free thiolate of the functionalization. The bounded antibodies were then released when the purified product was needed (FIG. 2). Research has already been focusing towards the use of membranes for separations as a cheaper solution compared to traditional methods. However, membrane separation has been limited to use only as a substitute for one step. Conversely, the membranes described herein not only achieve separation, but also the recovery and purification of antibodies. The selective functionalization of the polymeric membranes described herein retained only the desired antibody and later released it to obtain the pure product.

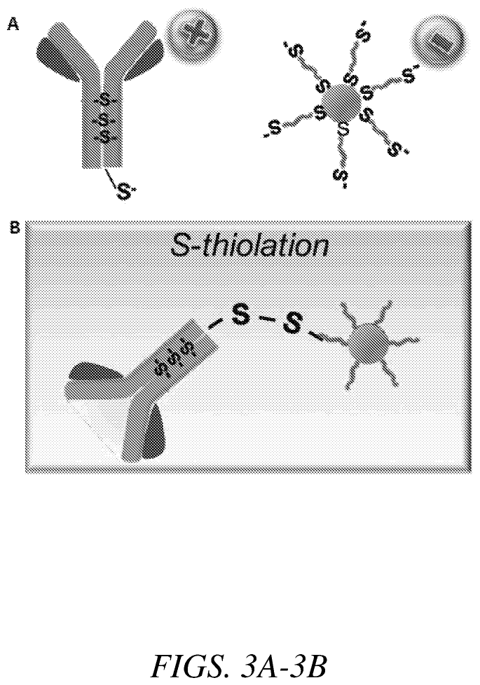

As is understood, no gold nanoparticle-functionalized membranes have been studied for separation processes. Gold nanoparticles were implemented to provide affinity, mainly because of their biocompatible and reversible binding behavior, together with their high surface area-to-volume ratio, which offered a large number of binding sites. Moreover, a second step of the functionalization included the incorporation of dithiol molecules that bind to the gold nanoparticles through one of the two thiol groups due to the strong interaction between gold and sulfur. However, the other end of the dithiol was exposed to the rest of the environment and was further used for reaction. In particular, the exposed end of the dithiol was thought to interact with the partially reduced thiolate groups of the heavy chain of the antibodies. This reaction is called S-thiolation and is known to be a redox reaction (FIGS. 3A-3B). This was the first time S-thiolation was used for the adsorption of IgG. The main advantage of dithiol was that it offered a specific binding with the antibody. For example, antibodies are characterized by having a Y-shape: on top of the Y there are the two active sites of the molecules, which cannot get in contact with any surface otherwise the overall activity is compromised. Therefore, the only way to achieve the adsorption of IgG was through the heavy chains (bottom of the Y), which around neural pH partially reduce to expose —SH sites. Besides the specificity of the binding, the membrane was highly selective, meaning that it only involved the antibodies, without promoting any interaction with the counter-protein, BSA, otherwise separation as well as purity would have been compromised.

Due to the innovative technology implemented, a new approach for desorption of IgG from the membrane was also developed. In particular, it was necessary to find an agent that cleaved disulfide bonds, breaking the bond between the antibody and the dithiol. In particular, dithiothreitol (DTT) was selected as the reducing agent for breaking disulfide bonds. Indeed, after its usage, concentration of the antibody in solution was detected.

### Materials and Methods

The polymer chosen for the membrane was polystyrene-b-poly-4-vinylpyridine (PS-b-P4VP). The separation process, as well as adsorption and desorption, were performed at a pH of about 6.5, such that the functionalized polymeric membrane and bovine serum albumin (BSA) were negatively charged (thus repelling each other and favoring BSA permeation) and IgG was positively charged (thus being attracted by the membrane). The operation pressure for the permeability tests was about 1 bar. The concentration of the IgG solution used for the adsorption tests was about 0.3 mg/mL. The concentration of the DTT solution used for the recovery step was about 0.1 mg/mL.

### Materials

The membrane was prepared based on Poly(styrene-b-4-vinylpyridine) (PS-b-P4VP), which was purchased from Polymer Source, Canada. Dimethyl formamide (DMF), 1,4-Dioxane (DOX) and acetone (Ac) were obtained from Sigma Aldrich. All chemicals were analytical reagents and were used without pre-treatment.

The antibodies used in the experiments were γ-globulins, IgG; the counter-protein to separate was bovine serum albumin, BSA. The biomolecules solutions were prepared in phosphate saline buffer (PBS). 1,3-propanedithiol, IgG, BSA and PBS were purchased from Sigma Aldrich.

### Membrane Preparation

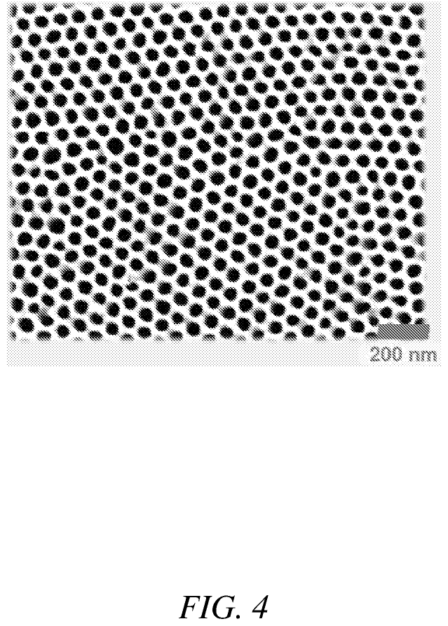

The preparation of isoporous membranes was based on self-assembly and non-solvent induced phase separation. A solution containing about 16 wt % PS-b-P4VP, about 24 wt % DMF, about 44 wt % DOX and about 16% Ac was cast on glass plates, the solvent was evaporated for about 20 s, followed by immersion in water. FIG. 4 provides an image of the polymeric membrane, according to one or more embodiments of the present disclosure.

### Synthesis of Gold Nanoparticles

The preparation of gold nanoparticles (Au NPs) included adding a 25 mM aqueous solution of tetrachloroauric (III) acid trihydrate (HAuCl₄×3H₂O) to about 500 mL of boiling water and, after few minutes, adding an aqueous solution with about 50 mM trisodium citrate tribasic dihydrate as the reducing and stabilizing agent. The reduced gold atoms precipitated and agglomerated, forming gold nanoparticles surrounded and stabilized by citrate molecules. By observing the UV-Vis spectra, the adsorption peak was localized at 524 nm, corresponding to a size of the nanoparticles of ˜20-25 nm. Moreover, by using Beer-Lambert law (A=ε·b·c), the concentration (c) of nanoparticles in the stock solution was calculated to be ˜1.5·10⁻¹⁰ M, using an extinction coefficient (ε) equal to 2.93·10⁹ M⁻¹cm⁻¹.

### Membrane Functionalization

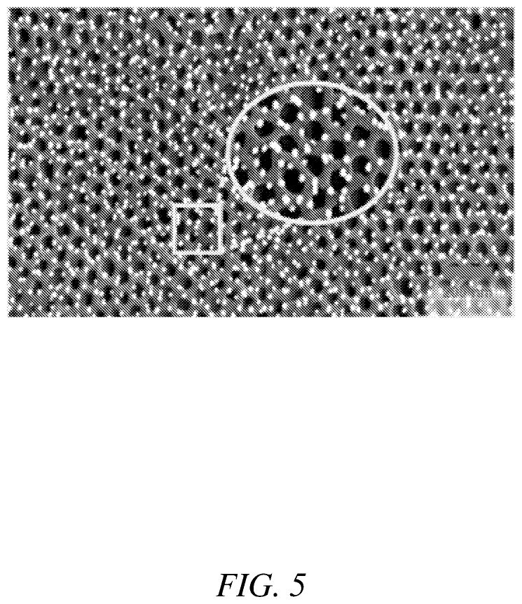

In a first step, the gold nanoparticles were first incorporated in the polymeric membranes. For that, the membrane was left in contact with an Au NPs solution for about 8 hours, under stirring conditions. In a second step, the nanoparticles were functionalized with dithiol molecules. Thiol effectively binded to AuNP. The incorporation of dithiols followed a similar approach, reported for nanoparticle functionalization. The gold-functionalized membrane was immersed in a solution of 1,3-propanedithiol in ethanol for hours, followed by rinsing with ethanol, in order to remove the unreacted thiol. FIG. 5 shows an image of the functionalized polymeric membrane, according to one or more embodiments of the present disclosure.

### Chemical and Morphological Characterization

The success of the functionalization, resulting from the incorporation of the gold nanoparticles and dithiol molecules, was monitored by Fourier Transform Infrared Spectroscopy (Thermo Scientific™ Nicolet iS10) at room temperature, using attenuated total reflectance (ATR).

The morphology was observed by scanning electron microscopy (SEM) on a Zeiss Merlin microscope with Gemini II column. The effect of the functionalization on the hydrophilicity of the membranes was characterized by measuring the contact angle of water on the surface of the membrane. The measurements were conducted on a Kruss Easydrop (Kruss GmbH, Germany) at room temperature.

### Separation and Recovery Experiments

The ability of the functionalized membrane to selectively separate the antibodies (IgG) from BSA was investigated. Ideally the system should be able to temporarily retain (i.e., adsorb) the antibody, and release it at a later controlled stage. For this reason, permeability and rejection tests were performed, along with adsorption tests. Permeance and proteins rejection were tested using a permeability line, under working conditions of about 1 bar. The variations in protein concentration (both for rejection and adsorption tests) were recorded by using a Spectrophotometer (NanoDrop 2000c). The amount of protein was quantified by calibrating the specific wavelength at which the biomolecules adsorb.

### Results

In this work, isoporous membranes functionalized with gold nanoparticles were prepared. The membrane was evaluated in two separation procedures: (i) size-driven separation and (ii) adsorption-desorption specific binding. First the membranes preparation and functionalization is discussed, followed by the performance evaluation in the separation methods.

### Membrane Preparation and Characterization

The Au NPs were chosen for the membrane functionalization, by taking into consideration the possibility of reversible attachment—detachment towards biological molecules. Hence, the first step of the process included obtaining a homogeneous distribution of gold nanoparticles on the surface of an isoporous PS-b-P4VP membrane. This goal was achieved through a batch functionalization, by membrane immersion in the Au NPs solution, as detailed above. Through SEM characterization, it was noticed that Au NPs decorated the pore walls of the membrane, pores which were rich in P4VP. Au NPs were preferentially placed in the P4VP phase of amphiphilic block copolymer systems. The incorporation of Au NPs with citrate ligands was confirmed visually, as the membrane changed color from white to purple after adding the Au NPs, and by FTIR spectroscopy. In particular, the enhancement of a broad —OH peak and —CH₂ peaks in the FTIR spectra demonstrated the presence of citrate ligands. After the incorporation of the gold nanoparticles, the second step was the functionalization with dithiol molecules. At this point, the FTIR spectra was particularly valuable to confirm the success, because no visual changes occurred after this functionalization. The relevant differences between unmodified and modified membranes happened in the area of the aliphatic vibrations, therefore in the region between 3050 and 2900 cm⁻¹. Two new peaks appeared after the reaction with the aliphatic dithiol. The dithiols strongly binded to functionalized gold nanoparticles.

1,3-propanedithiol was chosen in this work, because the goal was to attach one of the two thiol groups to the gold nanoparticles and have the other one free to react with the antibody and undergo S-thiolation. The main drawback of using a dithiol was that, in principle, both thiols of the same molecule could react with gold, thus inducing a phenomenon referred as “back-biting”. If this happens, no free —SH would be left for the antibody binding. Au NPs in this work were first embedded in the membrane pores. Their mobility was restricted after this first step. Back-biting would only occur if the dithiol length would be high enough. This was the case with the short 1,3-propanedithiol.

### Size-Driven Separation

The primary goal of the current work was the separation of antibodies (IgG) from other proteins, in this case BSA. In principle, IgG and BSA could be separated by simple size exclusion, because BSA is smaller than IgG (66.5 kDa vs 150 kDa) with diameters of 6 and 15 nm, respectively. This should be easily feasible if BSA did not have a specific interaction with the membrane and could fully permeate. Apart from the pore size, the surface charge is important for protein separations. The isoelectric points (IEP) of BSA and IgG are at pH=4.9 and pH-7, respectively. According to zeta potential measurements, the unmodified PS-b-P4VP membrane surface had a negative charge above pH=6.8. However, when functionalized with gold nanoparticles (which have a negative charge), the overall surface charge became negative, even at lower pH. Therefore, in order to minimize a non-specific adsorption between the membrane and BSA, the pH was set at 6.5. At this condition, the BSA and the membrane were negative.

Once the pH was set, the operational conditions (regarding flux/permeance) needed to be tested. The pH-responsive behavior of PS-b-P4VP has been extensively studied. It is known that the permeability only decreases at pH values lower than those applied in this investigation. It was confirmed that the permeation of the modified membrane for 1M phosphate saline buffer (PBS) with pH adjusted to 6.5 was around 800 L m⁻² h⁻¹ bar⁻¹, less than 10% lower than at pH=7.5. The BSA and IgG rejections were tested at pH=6.5. It was successfully recorded that BSA mostly permeated through the membrane (rejection was lower than 8%), whereas IgG is mostly retained (>90%), proving that the separation by size and charge for this system was feasible.

### Adsorption-Desorption Driven Separation

A main innovation of this work was the functionalization of isoporous polymeric membranes, providing specific biomolecules adsorption-desorption by covalent bonding and debonding. The adsorption was performed with the Au NPs/dithiol functionalized membranes, following an approach analogous to that traditionally adopted for ion-exchange chromatography: after 5 hours of equilibration in a PBS solution at pH=6.5, the membrane was immersed overnight in a solution of IgG (0.3 g/l), under stirring. After that, the membrane was quickly washed to remove any impurities or unbounded proteins. The final step was the elution or desorption to recover the antibody. However, despite the similarities of the protocol in the first stages, the main adsorption mechanism was not based on charge or acid-base interaction, like in the ion-exchange chromatography. The adsorption was promoted by a specific disulfide bond between the antibody and the modified membrane. The generated new bond could not be broken by regular elution techniques, normally used in ion exchange chromatography. Bearing this in mind, adsorption tests were performed at pH=6.5.

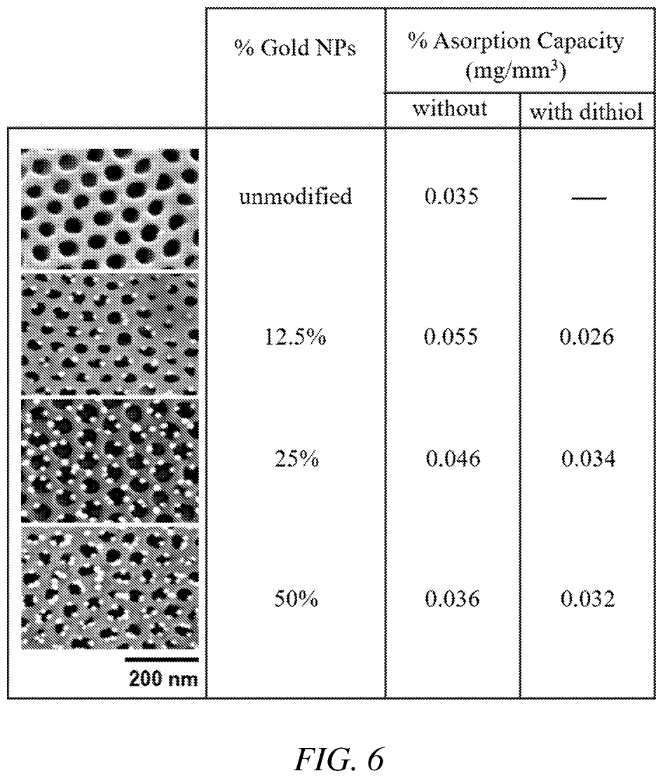

To study the effect of the functionalization on the adsorption, three membranes functionalized with different amounts of gold nanoparticles were prepared. For that, the pristine membranes were immersed in Au NPs solutions with different concentrations: 7.5, 3.8 and 1.9 10⁻¹¹ M. The effect of the gold nanoparticles with and without further functionalization with dithiol was compared. In the case of the complete functionalization (gold and dithiol), the concentration of 1,3-propanedithiol for functionalization was 200 mM and the exposure time of 6 hours. The results (average of 3 measurements) are reported in FIG. 6 regarding adsorption capacity, q′_{ADS}, calculated as the mass of antibody adsorbed per unit of volume (mg/mm³). Two opposite trends were observed, when the amount of gold nanoparticles in the solution is increasing: in the absence of dithiol, the adsorption capacity decreased, while in presence of dithiol it increased. The difference between the two conditions was just the dithiol, which was introduced in the system to promote selective adsorption of the antibody, forming a disulfide bond. In absence of dithiol, the antibody was still able to bind to the surface of the gold nanoparticles and of the membrane, but this adsorption was non-specific. It was found, moreover, that a substantial extent of adsorption took place even in the unmodified membrane, meaning that there were some non-specific adsorption sites within the PS-b-P4VP membrane. However, when the same membrane was functionalized with gold nanoparticles (without dithiol), the adsorption capacity decreased, meaning that some of the available binding sites in the pristine membrane were not active anymore. They were probably the pyridine groups in the membrane. When the gold nanoparticles were introduced, they interacted with pyridine, shielding its further interaction with the antibodies. In other words, the pyridine sites became less accessible for non-specific interaction with antibodies. In conclusion, to avoid non-specific binding, high amount of Au NPs should be used.

On the contrary, when the gold-functionalized membranes were further modified with dithiol molecules, the adsorption capacity increased when the Au NPs concentration increased from 1.9 to 3.8 10⁻¹¹ M. In this case, the increase in adsorption capacity was clearly due to the increase of available thiol groups for S-thiolation.

Summing the conclusions coming from these experiments, it can be claimed that the best adsorption (in quantity and specificity) was given by membranes functionalized with a high density of nanoparticles and dithiol. Indeed, not only was it possible to block the non-specific sites, but also to promote an increased number of binding sites through dithiol incorporation.

Once the specificity of the binding was proven, the next step involved proving its selectivity. In other words, the modification of the membrane was to enhance the adsorption of IgG, without promoting any interaction with BSA. The functionalized membrane in BSA solution was tested, and it was successfully proved that, after 24 hours, no adsorption was measured, confirming the selectivity of the functionalization towards the antibody only. Albumins can suffer S-thiolation with small thiol molecules. However the sulfhydryl groups in bovine albumin seemed not to be completely accessible for reaction with the thiol groups immobilized in the membrane. In this a selective binding of IgG was favored.

Once the antibody was attached to the membrane, for a successful full product purification and utilization, the final desorption step was fundamental. Due to the formation of the disulfide bond, traditional techniques implemented for the elution, such as a change in pH of the buffer or increase in the ionic strength of the solution, would not be enough to promote the desorption. The disulfide bonds between the antibody and the thiolate group of the dithiol would need to be broken. For this purpose, dithiothreitol (DTT), was used as a reducing agent, which effectively cleaved or broke the disulfide bonds.

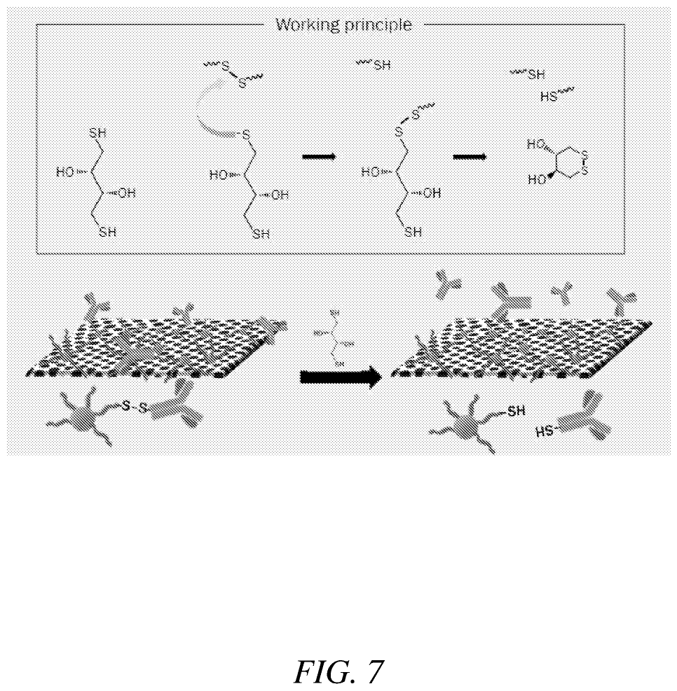

Practically, the membrane with adsorbed antibodies was immersed in a diluted DTT solution (0.1 mg/mL) overnight, and the IgG concentration in solution was monitored by UV absorption measurements. After ˜20 hours, the antibody was detected in solution and the recovered amount was quantified between 40 to 50% of the initially adsorbed value. The yield was enhanced by process optimization. Optimization of the DTT concentration and immersion time increased the desorption. pH variation was useful in the case of partially unspecific electrostatic adsorption. FIG. 7 is a schematic diagram illustrating the desorption process using DTT, according to one or more embodiments of the present disclosure.

Other embodiments of the present disclosure are possible. Although the description above contains much specificity, these should not be construed as limiting the scope of the disclosure, but as merely providing illustrations of some of the presently preferred embodiments of this disclosure. It is also contemplated that various combinations or sub-combinations of the specific features and aspects of the embodiments may be made and still fall within the scope of this disclosure. It should be understood that various features and aspects of the disclosed embodiments can be combined with or substituted for one another in order to form various embodiments. Thus, it is intended that the scope of at least some of the present disclosure should not be limited by the particular disclosed embodiments described above.

Thus the scope of this disclosure should be determined by the appended claims and their legal equivalents. Therefore, it will be appreciated that the scope of the present disclosure fully encompasses other embodiments which may become obvious to those skilled in the art, and that the scope of the present disclosure is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims.

The foregoing description of various preferred embodiments of the disclosure have been presented for purposes of illustration and description. It is not intended to be exhaustive or to limit the disclosure to the precise embodiments, and obviously many modifications and variations are possible in light of the above teaching. The example embodiments, as described above, were chosen and described in order to best explain the principles of the disclosure and its practical application to thereby enable others skilled in the art to best utilize the disclosure in various embodiments and with various modifications as are suited to the particular use contemplated. It is intended that the scope of the disclosure be defined by the claims appended hereto

Various examples have been described. These and other examples are within the scope of the following claims.

1. A functionalized polymeric membrane, comprising:
one or more dithiol compounds that extend from a nanoparticle provided on or near a surface and/or pores of a polymer material;
wherein at least one thiol of the dithiol compound binds to the nanoparticle and at least one thiol of the dithiol compound is a free thiol.

2. The membrane wherein the dithiol compounds include one or more of 1,2-ethanedithiol, butanedithiol, 1,3-propanedithiol, 1,5-pentanedithiol, 2,3-dimercapto-1-propanol, dithioerythritol, 3,6-dioxa-1,8-octanedithiol, 1,8-octanedithiol hexanedithiol, dithiodiglycol, pentanedithiol, decanedithiol, 2-methyl 1,4 butanedithiol, bis-mercaptoethylphenyl methane, 1,9-nonanedithiol(1,9-dimercaptononane), glycol dimercaptoacetate, 1,2-benzenedithiol, 1,3-benzenedithiol, 1,4-benzenedithiol, 2,4,6-trimethyl-1,3-benzenedimethanethiol, durene-α1, α2-dithiol, 3,4-dimercaptotoluene, 4-methyl-1,2-benzenedithiol, 2,5-dimercapto-1,3,4-thiadiazole, 4,4′-thiobisbezenedithiol, and bis(4-mercaptophenyl)-2,2′-propane(bisphenol dithiol).

3. The membrane of claim 1, wherein the nanoparticle is a plurality of nanoparticles.

4. The membrane of claim 1, where the nanoparticle is one or more of a gold nanoparticle, a silver nanoparticle, and a copper nanoparticle.

5. The membrane of claim 1, wherein the polymer material includes a porous support layer and an isoporous layer.

6. The membrane of claim 1, wherein the polymer material includes a block copolymer.

7. The membrane of claim 1, wherein the polymer material includes one or more of polystyrene, poly-4-vinylpyridine, poly-2-vinylpyridine, polybutadiene, polyisoprene, poly(ethylene-stat-butylene), poly(ethylene-alt-propylene), polysiloxane, polyalkyleneoxide, poly-F-caprolactone, polylactide, polyalkylmethacrylate, polymethacrylic acid, polyalkylacrylate, polyacrylic acid, polyhydroxyethylmethacrylate, polyacrylamide, poly-N-alkylacrylamide, polysulfone, polyaniline, polypyrrole, polytriazole, polyvinylimidazole, polytetrazole, polyethylenediamine, polyvinylalcohol, polyvinylpyrrolidone, polyoxadiazole, polyvinylsulfonic acid, polyvinylphosphonic acid, and polymers with quarternary ammonium groups.

8. The membrane of claim 1, wherein the free thiol of the dithiol compound selectively binds to an antibody.

9. The membrane of claim 1, wherein the free thiol of the dithiol compound specifically binds to a thiol of a heavy chain in the Fc region of the antibody.

10. A method of separating and/or recovering a purified antibody, comprising:
contacting a feed stream containing an antibody and other biomolecules with a functionalized polymeric membrane to separate the antibody from the feed stream; and
applying a reducing agent to release the antibody from the membrane and recover a purified antibody;
wherein the functionalized polymeric membrane includes a plurality of free thiols selective to binding the antibody.

11. The method of claim 10, wherein the contacting proceeds at about a neutral pH.

12. The method of claim 10, wherein the feed stream includes one or more of proteins, peptides, cells, cell debris, nucleic acids, endotoxins, and viruses.

13. The method of claim 10, wherein the antibody is separated from the feed stream based on one or more of adsorption, size exclusion, and affinity.

14. The method of claim 10, wherein the free thiol specifically binds to a thiol of a heavy chain in the Fc region of the antibody.

15. The method of claim 10, wherein the free thiol is selective to binding the antibody to the exclusion or substantial exclusion of the other biomolecules.

16. The method of claim 10, wherein the free thiol of the membrane binds to the thiol of the antibody through formation of a disulfide bond.

17. The method of claim 10, wherein the other biomolecules permeate through the functionalized polymeric membrane.

18. The method of claim 10, wherein the reducing agent cleaves a disulfide bond between the membrane and the antibody to release the antibody.

19. The method of claim 10, wherein the purified antibody is recovered without degrading an activity of the antibody.

20. A functionalized polymeric membrane, comprising:
one or more dithiol compounds that extend from a gold nanoparticle provided on a surface of a block copolymer membrane;
wherein at least one thiol of the dithiol compound binds to the nanoparticle and at least one thiol of the dithiol compound is a free thiol.
